(19) United States
(12) Patent Application Publication
(10) Pub. No.: US 2019/0099374 A1
(43) Pub. Date: Apr. 4, 2019

(54) STABLE FORMULATIONS FOR LYOPHILIZING THERAPEUTIC PARTICLES

(71) Applicant: PFIZER INC., New York, NY (US)

(72) Inventors: Ujjwal Joshi, Waltham, MA (US); Susan Low, Pepperel, MA (US); Young-Ho Song, Natick, MA (US); Jeanne Tran, Dorchester, MA (US); Greg Troiano, Pembroke, MA (US)

(73) Assignee: PFIZER INC., New York, NY (US)

(21) Appl. No.: 16/086,717

(22) PCT Filed: Mar. 16, 2017
(86) PCT No.: PCT/IB2017/051543

§ 371 Date: Sep. 20, 2018

Related U.S. Application Data
(60) Provisional application No. 62/461,410, filed on Feb. 21, 2017.
(60) Provisional application No. 62/313,436, filed on Mar. 25, 2016.

(51) Int. Cl. A61K 9/19; A61K 9/00; A61K 9/51

(52) U.S. Cl. CPC A61K 9/19; A61K 9/0019; A61K 31/337; A61K 9/5123; A61K 9/5161; A61K 9/5153

(57) ABSTRACT

The present disclosure generally relates to lyophilized pharmaceutical compositions comprising polymeric nanoparticles which, upon reconstitution, have low levels of greater than 10 micron size particles. Other aspects of the invention include methods of making such nanoparticles.

This application is a national phase filing under 35 U.S.C. § 371 of international patent application number PCT/IB2017/051543 filed Mar. 16, 2017, which in turn claims the benefit of priority to U.S. Provisional Patent Application Ser. No. 62/313,436 filed Mar. 25, 2016 and to U.S. Provisional Patent Application Ser. No. 62/461,410 Feb. 21, 2017, the disclosure of each of these applications is hereby incorporated by reference in its entirety.

### BACKGROUND

Systems that deliver certain drugs to a patient (e.g., targeted to a particular tissue or cell type or targeted to a specific diseased tissue but not normal tissue), or that control release of drugs has long been recognized as beneficial.

For example, therapeutics that include an active drug and that are, e.g., targeted to a particular tissue or cell type or targeted to a specific diseased tissue but not to normal tissue, may reduce the amount of the drug in body tissues that do not require treatment. This is particularly important when treating a condition such as cancer where it is desirable that a cytotoxic dose of the drug is delivered to cancer cells without killing the surrounding non-cancerous tissue. Further, such therapeutics may reduce the undesirable and sometimes life-threatening side effects common in anticancer therapy. In addition, such therapeutics may allow drugs to reach certain tissues they would otherwise be unable to reach.

Delivery of therapeutic nanoparticles can be achieved through parenteral injection of a reconstituted suspension of the nanoparticles. The original nanoparticle suspension is lyophilized, i.e., freeze dried, for storage before reconstitution. Freeze drying a nanoparticle suspension potentially creates a product for reconstitution with far superior storage stability than its frozen suspension counterpart. Further, freeze drying may provide easier storage that may not require constant, very low, temperatures. However, the reconstituted lyophilisate must possess physicochemical and performance attributes that are comparable or superior to the original suspension. Redispersing into particles of the same size without trace particulates due to micro-aggregation or undispersed particles is the most challenging aspect of nanoparticle suspension lyophilization.

Accordingly, a need exists for nanoparticle therapeutics and methods of making such nanoparticles, that are capable of delivering therapeutic levels of drug to treat diseases such as cancer, and possess superior storage capabilities.

### SUMMARY

In one aspect, a lyophilized pharmaceutical composition is provided. The lyophilized pharmaceutical composition comprises polymeric nanoparticles comprising: a poly(lactic) acid-block-poly(ethylene)glycol copolymer or poly(lactic)-co-poly(glycolic) acid-block-poly(ethylene)glycol copolymer, and a therapeutic agent; a sugar alcohol; and a cyclodextrin.

In certain embodiments, the weight ratio of the polymeric nanoparticles: the sugar alcohol: the cyclodextrin is 0.2-1.0:1.0:0.5-1.8; 0.3-0.9:1.0:0.6-1.6; 0.4-0.9:1.0:0.6-1.4; 0.4-0.9:1.0:0.8-1.2; 0.5-0.8:1.0:0.8-1.2; 0.5-0.7:1.0:0.8-1.2; or 0.5-0.7:1.0:0.9-1.1. In some further embodiments, the sugar alcohol comprises mannitol; the cyclodextrin comprises hydroxypropyl β-cyclodextrin; and the polymeric nanoparticles comprises about 5-20 weight % of the therapeutic agent.

In certain embodiments, upon reconstitution of a contemplated lyophilized pharmaceutical composition in an aqueous medium, the composition comprises about 2 to about 12 weight percent of the sugar alcohol (e.g. mannitol); and about 2 to about 12 weight percent of the cyclodextrin (e.g. hydroxypropyl β-cyclodextrin). In certain embodiments, the reconstituted composition comprises about 6 to about 10 weight percent of the sugar alcohol. In certain embodiments, the reconstituted composition comprises about 6 to about 9 weight percent of the cyclodextrin. In certain embodiments, the reconstituted composition comprises about 6 to about 10 (e.g. about 6 to about 9; or about 7 to about 8) weight percent of the sugar alcohol (e.g. mannitol); about 6 to about 9 (e.g. about 6 to about 8; or about 7 to about 8) weight percent of the cyclodextrin (e.g. hydroxypropyl β-cyclodextrin); and about 10 to about 100 mg/mL concentration of the polymeric nanoparticles (e.g. about 20 to about 90 mg/mL; 20 to about 80 mg/mL; about 30 to about 80 mg/mL; about 40 to about 80 mg/mL; about 40 to about 70 mg/mL; about 40 to about 60 mg/mL; about 40 to about 50 mg/mL; about 50 to about 60 mg/mL; about 40 mg/mL; about 45 mg/mL; about 50 mg/mL; or about 55 mg/mL). In certain embodiments, the composition comprises about 7.5 weight percent cyclodextrin and about 7.5 weight percent sugar alcohol.

In certain embodiments, the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and mixtures thereof. In certain embodiments, the sugar alcohol is mannitol.

In certain embodiments, the cyclodextrin is selected from the group consisting of α-cyclodextrin, β-cyclodextrin, γ-cyclodextrin, and mixtures thereof. In certain embodiments, the cyclodextrin is hydroxypropyl β-cyclodextrin.

In certain embodiments, a reconstituted composition comprises about 7.5 weight percent hydroxypropyl β-cyclodextrin and about 7.5 weight percent mannitol.

In certain embodiments, the poly(lactic) acid portion of the poly(lactic) acid-block-poly(ethylene)glycol copolymer has a weight average molecular weight of about 10 kDa to about 25 kDa and the poly(ethylene)glycol portion of the poly(lactic) acid-block-poly(ethylene)glycol copolymer has a weight average molecular weight of about 4 to about 6 kDa. In certain embodiments, the poly(lactic) acid portion of the poly(lactic) acid-block-poly(ethylene)glycol copolymer has a weight average molecular weight of about 16 kDa and the poly(ethylene)glycol portion of the poly(lactic) acid-block-poly(ethylene)glycol copolymer has a weight average molecular weight of about 5 kDa.

In certain embodiments, contemplated polymeric nanoparticles have a diameter of about 60 nm to about 140 nm, or about 80 nm to about 120 nm.

In certain embodiments, contemplated polymeric nanoparticles comprise about 3 to about 40 weight percent therapeutic agent.

In certain embodiments, contemplated polymeric nanoparticles further comprise a ligand conjugated polymer.

In certain embodiments, the therapeutic agent is selected from the group consisting of a taxane, an epothilone, an mTOR inhibitor, a vinca alkaloid, a diterpene derivative, and an alkylating agent.

In certain embodiments, a contemplated composition is capable of being reconstituted in about 30 to about 120 seconds, or about 40 to about 90 seconds.

In certain embodiments, upon reconstitution of a contemplated lyophilized pharmaceutical composition in less than or about 100 mL of an aqueous medium, the reconstituted lyophilized pharmaceutical composition comprises less than 3000 particles having a size greater than or equal to 10 microns.

In certain embodiments, upon reconstitution of a contemplated lyophilized pharmaceutical composition in less than or about 100 mL of an aqueous medium, the reconstituted lyophilized pharmaceutical composition comprises less than 300 particles having a size greater than or equal to 25 microns.

In certain embodiments, upon reconstitution of a contemplated lyophilized pharmaceutical composition in less than or about 100 mL of an aqueous medium, the reconstituted lyophilized pharmaceutical composition comprises less than 600 particles having a size greater than or equal to 25 microns and/or less than 6000 particles having a size greater than or equal to 10 microns.

In certain embodiments, upon reconstitution of a contemplated lyophilized pharmaceutical composition in less than or about 100 mL of an aqueous medium, the reconstituted lyophilized pharmaceutical composition comprises less than 0.05 weight percent of particles having a size between 5 and 50 microns.

In certain embodiments, upon reconstitution of a contemplated lyophilized pharmaceutical composition in less than or about 10 mL of an aqueous medium, the reconstituted lyophilized pharmaceutical composition comprises: 40 mg/mL or more concentration of polymeric nanoparticles comprising a poly(lactic) acid-block-poly(ethylene)glycol copolymer and a therapeutic agent; about 6 to about 9 weight percent of mannitol; and about 6 to about 9 weight percent of a cyclodextrin; wherein the reconstituted lyophilized pharmaceutical composition comprises less than 600 particles having a size greater than or equal to 10 microns.

In certain embodiments, a contemplated lyophilized pharmaceutical composition is lyophilized in 2.5 days or less, as compared to a lyophilized composition that does not contain mannitol. In certain embodiments, the lyophilized pharmaceutical composition of the invention is lyophilized in a much shorter cycle time as compared in another lyophilized composition that does not contain mannitol (for example, a lyophilized composition comprising sucrose and HPbCD).

In another aspect, a lyophilized pharmaceutical dose is provided. The lyophilized pharmaceutical dose comprises polymeric nanoparticles comprising: a poly(lactic) acid-block-poly(ethylene)glycol copolymer or poly(lactic)-co-poly(glycolic) acid-block-poly(ethylene)glycol copolymer, and docetaxel, wherein the dose comprises about 30-37 mg, or about 33 mg, of the docetaxel; mannitol; and hydroxypropyl β-cyclodextrin.

In certain embodiments, a container is provided comprising about 6 mL to about 7 mL, or about 6.6 mL, of a contemplated lyophilized pharmaceutical dose. In certain embodiments, when the lyophilized pharmaceutical dose is reconstituted to a reconstituted dose of 13.2 mL, the container has less than about 454 particles having a size greater than or equal to 10 microns per mL of the reconstituted dose, and/or less than about 45 particles having a size greater than or equal to 10 microns per mL of the reconstituted dose.

In yet another aspect, a reconstituted lyophilized pharmaceutical composition suitable for parenteral administration is provided. The reconstituted lyophilized pharmaceutical composition comprises a 10-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a therapeutic agent; about 6 to about 10 weight percent of mannitol; and about 6 to about 9 weight percent of hydroxypropyl β-cyclodextrin.

In certain embodiments, a contemplated reconstituted lyophilized pharmaceutical composition comprises: less than 6000 microparticles of greater than or equal to 10 microns; and less than 600 microparticles of greater than or equal to 25 microns; per sample container having less than or about 100 mL of the composition.

In certain embodiments, a contemplated reconstituted lyophilized pharmaceutical composition comprises: less than 600 microparticles per mL of greater than or equal to 10 microns; and less than 60 microparticles per mL of greater than or equal to 25 microns.

In certain embodiments, a contemplated reconstituted lyophilized pharmaceutical composition comprises: less than 600 microparticles of greater than or equal to 10 microns; and less than 60 microparticles of greater than or equal to 25 microns; per sample container having less than or about 100 mL of the composition.

In certain embodiments, the poly(lactic) acid portion of the copolymer has a weight average molecular weight of about 16 kDa and the poly(ethylene)glycol portion of the copolymer has a weight average molecular weight of about 5 kDa.

In still another aspect, a method of preparing a lyophilized pharmaceutical composition suitable for parenteral administration upon reconstitution is provided. The method comprises providing a formulation comprising polymeric nanoparticles, wherein the polymeric nanoparticles comprise a therapeutic agent and a polymer selected from the group consisting of poly(lactic) acid-block-poly(ethylene)glycol copolymer and poly(lactic)-co-poly(glycolic) acid-block-poly(ethylene)glycol copolymer; adding hydroxypropyl β-cyclodextrin and mannitol to the formulation to form a pre-lyophilization aqueous formulation; and lyophilizing the pre-lyophilization aqueous formulation to form the lyophilized pharmaceutical formulation. In certain embodiments, the pre-lyophilization aqueous formulation comprises about 6 to about 10 (e.g. about 6 to about 9, about 6 to about 8, or about 7 to about 8) weight percent of mannitol, about 6 to about 9 (e.g. about 6 to about 8, or about 7 to about 8) weight percent of hydroxypropyl β-cyclodextrin, and about 10 to about 100 mg/mL concentration of the polymeric nanoparticles (e.g. about 20 to about 90 mg/mL, 20 to about 80 mg/mL, about 30 to about 80 mg/mL, about 40 to about 80 mg/mL, about 40 to about 70 mg/mL, about 40 to about 60 mg/mL, about 40 to about 50 mg/mL, about 50 to about 60 mg/mL, about 40 mg/mL, about 45 mg/mL, about 50 mg/mL, or about 55 mg/mL). In certain embodiments, the pre-lyophilization aqueous formulation comprises comprises about 6 to about 9 weight percent of mannitol, about 6 to about 9 weight percent of hydroxypropyl β-cyclodextrin, and about 10 to about 100 mg/mL concentration of the polymeric nanoparticles (e.g. about 20 to about 90 mg/mL, 20 to about 80 mg/mL, about 30 to about 80 mg/mL, about 40 to about 80 mg/mL, about 40 to about 70 mg/mL, about 40 to about 60 mg/mL, about 40 to about 50 mg/mL, about 50 to about 60 mg/mL, about 40 mg/mL, about 45 mg/mL, about 50 mg/mL, or about 55 mg/mL). In certain embodiments, the pre-lyophilization aqueous formulation comprises comprises about 6 to about 9 weight percent of mannitol, about 6 to about 9 weight percent of hydroxypropyl β-cyclodextrin, and about 30 to about 80 mg/mL concentration of the polymeric nanoparticles [e.g. about 6 to about 9 weight percent of mannitol, about 6 to about 8 weight percent of hydroxypropyl β-cyclodextrin, and about 40 to about 70 mg/mL concentration of the polymeric nanoparticles; about 6 to about 8 weight percent of mannitol, about 6 to about 8 weight percent of hydroxypropyl β-cyclodextrin, and about 40 to about 70 mg/mL concentration of the polymeric nanoparticles; or about 7 to about 8 weight percent of mannitol, about 7 to about 8 weight percent of hydroxypropyl β-cyclodextrin, and about 40 to about 60 mg/mL concentration of the polymeric nanoparticles; or about 7.5 weight percent of mannitol, about 7.5 weight percent of hydroxypropyl β-cyclodextrin, and about 40 to about 60 mg/mL concentration of the polymeric nanoparticles].

In certain embodiments, lyophilizing a contemplated pre-lyophilization aqueous formulation comprises a lyophilization cycle of about 4.5 days or less; or about 4 days or less; or about 3 days or less; or about 2.5 days; or about 60 hours.

In certain embodiments, lyophilizing a contemplated pre-lyophilization aqueous formulation comprises: loading the formulation in a lyophilizer at about 4° C.; first decreasing the lyophilizer temperature to about −45° C. and holding at about −45° C. for about 2 hours; warming the lyophilizer to about −12° C. and holding at about −12° C. for about 3 hours; second decreasing the lyophilizer temperature to about −45° C. and holding at about −45° C. for about 2 hours.

In certain embodiments, lyophilizing a contemplated pre-lyophilization aqueous formulation further comprises increasing the lyophilizer temperature to about 35° C. after the second decreasing step.

In certain embodiments, the lyophilizer pressure is about 250 mTorr.

In certain embodiments, lyophilizing a contemplated pre-lyophilization aqueous formulation comprises: loading the formulation in a lyophilizer at about 4° C.; first decreasing the lyophilizer temperature to at least about 10° C. less than a glass transition temperature of the sugar alcohol; warming the lyophilizer to at least about 10° C. more than the glass transition temperature of the sugar alcohol; and second decreasing the lyophilizer temperature to at least about 10° C. less than the glass transition temperature of the sugar alcohol.

In certain embodiments, lyophilizing a contemplated pre-lyophilization aqueous formulation further comprises increasing the lyophilizer temperature to about 1° C. more than a lyophilization cake collapse temperature of a contemplated lyophilized pharmaceutical composition.

### BRIEF DESCRIPTION OF THE DRAWINGS

FIG. 1 is a flow chart for an emulsion process for forming disclosed nanoparticles.

FIGS. 2A and 2B show a flow diagram for a disclosed emulsion process.

FIG. 3 depicts nanoparticle sizes (measured using DLS) of the various reconstituted nanoparticle suspensions disclosed herein.

FIG. 4 depicts the particulate counts of various reconstituted nanoparticle suspensions disclosed herein.

FIG. 5 depicts the particulate counts of various reconstituted nanoparticle suspensions disclosed herein.

FIG. 6 depicts nanoparticle sizes (measured using DLS) of the various reconstituted nanoparticle suspensions disclosed herein.

FIG. 7 depicts the particulate counts of various reconstituted nanoparticle suspensions disclosed herein.

FIG. 8 depicts the particulate counts of various reconstituted nanoparticle suspensions disclosed herein.

FIG. 9 depicts in vitro release of docetaxel of various nanoparticle suspensions disclosed herein.

FIG. 10 depicts non-annealed differential scanning calorimetry (DSC) properties of nanoparticle suspensions having 7.5% mannitol and 7.5% hydroxypropyl-β-cyclodextrin.

FIG. 11 depicts annealed differential scanning calorimetry (DSC) properties of nanoparticle suspensions having 7.5% mannitol and 7.5% hydroxypropyl-β-cyclodextrin.

FIG. 12 shows shelf temperature and vacuum pressure as a function of time for a lyophilization cycle, according to an embodiment.

### DETAILED DESCRIPTION

Described herein are lyophilized polymeric nanoparticle compositions, and methods of making and using such therapeutic compositions. Such compositions may be reconstituted from a lyophilized composition, and may include minimal large aggregations of nanoparticles and/or other materials. Disclosed compositions therefore may be suitable for parenteral use.

In certain embodiments, contemplated formulations containing a sugar alcohol and a cyclodextrin advantageously can be lyophilized significantly faster as compared to prior art formulations, while substantially retaining quality attributes. In some embodiments, reconstituted lyophilized formulations may exhibit advantageous properties such as, e.g., minimal micro-aggregation of nanoparticles and/or substantially similar release properties as compared to formulations prepared without a sugar alcohol and a cyclodextrin.

### Lyophilized Pharmaceutical Compositions

In some embodiments, a composition suitable for freezing is contemplated, which may include nanoparticles disclosed herein and a solution suitable for freezing. For example, in some embodiments, the solution may comprise water, a sugar alcohol, and a cyclodextrin. Without wishing to be bound by any theory, it is believed that the sugar alcohol may act, e.g., as a cryoprotectant and the cyclodextrin may act, e.g., as a lyoprotectant.

In certain embodiments, the sugar alcohol may be derived from a mono-, di-, or poly-saccharide, e.g., by reducing a sugar. In some embodiments, the sugar alcohol may be selected from the group consisting of glycerol, erythritol, threitol, arabitol, xylitol, ribitol, mannitol, sorbitol, galactitol, fucitol, iditol, inositol, volemitol, isomalt, maltitol, lactitol, and mixtures thereof.

In some embodiments, the sugar alcohol may be added to a suspension of nanoparticles at any concentration suitable for preparing a lyophilized composition. For example, in some instances, the concentration of the sugar alcohol in a suspension of nanoparticles may be between about 1 and about 15 weight percent, between about 1 and about 12 weight percent, between about 1 and about 10 weight percent, between about 1 and about 8 weight percent, between about 1 and about 6 weight percent, between about 1 and about 4 weight percent, between about 2 and about 12 weight percent, between about 2 and about 10 weight percent, between about 2 and about 8 weight percent, between about 2 and about 6 weight percent, between about 3 and about 12 weight percent, between about 3 and about 10 weight percent, between about 3 and about 8 weight percent, between about 3 and about 6 weight percent, between about 4 and about 12 weight percent, between about 4 and about 10 weight percent, between about 4 and about 8 weight percent, between about 4 and about 6 weight percent, between about 5 and about 15 weight percent, between about 5 and about 12 weight percent, between about 5 and about 10 weight percent, between about 5 and about 8 weight percent, between about 6 and about 15 weight percent, between about 6 and about 12 weight percent, between about 6 and about 10 weight percent, between about 6 and about 9 weight percent, between about 7 and about 15 weight percent, between about 7 and about 12 weight percent, between about 7 and about 10 weight percent, between about 7 and about 9 weight percent, between about 8 and about 15 weight percent, between about 8 and about 12 weight percent, between about 8 and about 10 weight percent, between about 9 and about 15 weight percent, between about 9 and about 12 weight percent, between about 10 and about 15 weight percent, or between about 10 and about 12 weight percent. In certain embodiments, the concentration of the sugar alcohol in a suspension of nanoparticles may be about 7.5 weight percent.

In some embodiments, the cyclodextrin may include α-cyclodextrin, β-cyclodextrin, γ-cyclodextrin, or mixtures thereof. Non-limiting exemplary cyclodextrins contemplated for use in the compositions disclosed herein include hydroxypropyl-β-cyclodextrin (HPbCD), hydroxyethyl-β-cyclodextrin, sulfobutylether-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-β-cyclodextrin, glycosyl-β-cyclodextrin, and maltosyl-β-cyclodextrin.

In certain embodiments, the cyclodextrin may be added to a suspension of nanoparticles at any concentration suitable for preparing a lyophilized composition. For example, in some instances, the concentration of the cyclodextrin in a suspension of nanoparticles may be between about 1 and about 15 weight percent, between about 1 and about 12 weight percent, between about 1 and about 10 weight percent, between about 1 and about 8 weight percent, between about 1 and about 6 weight percent, between about 1 and about 4 weight percent, between about 2 and about 12 weight percent, between about 2 and about 10 weight percent, between about 2 and about 8 weight percent, between about 2 and about 6 weight percent, between about 3 and about 12 weight percent, between about 3 and about 10 weight percent, between about 3 and about 8 weight percent, between about 3 and about 6 weight percent, between about 4 and about 12 weight percent, between about 4 and about 10 weight percent, between about 4 and about 8 weight percent, between about 4 and about 6 weight percent, between about 5 and about 15 weight percent, between about 5 and about 12 weight percent, between about 5 and about 10 weight percent, between about 5 and about 8 weight percent, between about 6 and about 15 weight percent, between about 6 and about 12 weight percent, between about 6 and about 10 weight percent, between about 6 and about 9 weight percent, between about 7 and about 15 weight percent, between about 7 and about 12 weight percent, between about 7 and about 10 weight percent, between about 7 and about 9 weight percent, between about 8 and about 15 weight percent, between about 8 and about 12 weight percent, between about 8 and about 10 weight percent, between about 9 and about 15 weight percent, between about 9 and about 12 weight percent, between about 10 and about 15 weight percent, or between about 10 and about 12 weight percent. In certain embodiments, the concentration of the cyclodextrin in a suspension of nanoparticles may be about 7.5 weight percent.

The present disclosure relates in part to lyophilized pharmaceutical compositions that, when reconstituted, has a minimal amount of large aggregates. Such large aggregates may have a size greater than about 0.5 μm, greater than about 1 μm, or greater than about 10 μm, and can be undesirable in a reconstituted solution. Aggregate sizes can be measured using a variety of techniques including those indicated in the U.S. Pharmacopeia at 32 <788>, hereby incorporated by reference. The tests outlined in USP 32 <788> include a light obscuration particle count test, microscopic particle count test, laser diffraction, and single particle optical sensing. In one embodiment, the particle size in a given sample is measured using laser diffraction and/or single particle optical sensing.

The USP 32 <788> by light obscuration particle count test sets forth guidelines for sampling particle sizes in a suspension. For solutions with less than or equal to 100 mL, the preparation complies with the test if the average number of particles present does not exceed 6000 per container that are ≥10 μm and 600 per container that are ≥25 μm.

As outlined in USP 32 <788>, the microscopic particle count test sets forth guidelines for determining particle amounts using a binocular microscope adjusted to 100±10× magnification having an ocular micrometer. An ocular micrometer is a circular diameter graticule that consists of a circle divided into quadrants with black reference circles denoting 10 μm and 25 μm when viewed at 100× magnification. A linear scale is provided below the graticule. The number of particles with reference to 10 μm and 25 μm are visually tallied. For solutions with less than or equal to 100 mL, the preparation complies with the test if the average number of particles present does not exceed 3000 per container that are ≥10 μm and 300 per container that are ≥25 μm.

Dynamic light scattering (DLS) may be used to measure particle size, but it relies on Brownian motion so the technique may not detect some larger particles. Laser diffraction relies on differences in the index of refraction between the particle and the suspension media. The technique is capable of detecting particles at the sub-micron to millimeter range. Relatively small (e.g., about 1-5 weight %) amounts of larger particles can be determined in nanoparticle suspensions. Single particle optical sensing (SPOS) uses light obscuration of dilute suspensions to count individual particles of about 0.5 μm. By knowing the particle concentration of the measured sample, the weight percentage of aggregates or the aggregate concentration (particles/mL) can be calculated.

Reconstitution shows equivalent DLS size distributions when compared to the starting suspension. However, laser diffraction can detect particles of >10 μm in size in some reconstituted solutions. Further, SPOS also may detect >10 μm sized particles at a concentration above that of the FDA guidelines (10⁴-10⁵ particles/mL for >10 μm particles).

In some embodiments, a 10 mL aqueous sample of a disclosed composition upon reconstitution comprises less than 600 particles per mL having a size greater than or equal to 10 microns; and/or less than 60 particles per mL having a size greater than or equal to 25 microns. In some embodiments, a 10 mL aqueous sample of a disclosed composition upon reconstitution comprises less than 500 particles per mL, less than 400 particles per mL, less than 300 particles per mL, less than 200 particles per mL, less than 100 particles per mL, less than 80 particles per mL, less than 60 particles per mL, or less than 40 particles per mL having a size greater than or equal to 10 microns. In certain embodiments, a 10 mL aqueous sample of a disclosed composition upon reconstitution comprises less than 50 particles per mL, less than 40 particles per mL, less than 30 particles per mL, less than 20 particles per mL, less than 10 particles per mL, or less than 5 particles per mL having a size greater than or equal to 25 microns.

In one aspect, the invention provides a lyophilized pharmaceutical composition comprising polymeric nanoparticles, wherein upon reconstitution of the lyophilized pharmaceutical composition at a nanoparticle concentration of about 50 mg/mL, or about 40 mg/mL, in less than or about 13.2 mL of an aqueous medium, the reconstituted composition suitable for parenteral administration comprises less than 6000, less than 5000, less than 4000, less than 3000, less than 2000, less than 1500, or less 1000 microparticles of greater than or equal to 10 microns; and/or less than 600, less than 500, less than 400, less than 300, or less than 250 microparticles of greater than or equal to 25 microns.

The reconstituted composition may have minimal aggregation as compared to a reconstituted composition that does not contain a sugar alcohol and/or a cyclodextrin. In some embodiments, the reconstituted composition may have a polydispersity index of less than 0.2.

In some embodiments, reconstitution of the lyophilized pharmaceutical composition in less than or about 100 mL of an aqueous medium results in a reconstituted lyophilized pharmaceutical composition comprising less than 0.05 weight percent of particles having a size between 5 and 50 microns.

In some embodiments, a contemplated lyophilized composition may be reconstituted quickly, which, for example, may be advantageous, e.g., in a clinical setting when preparing the composition for administration. For instance, in some embodiments, a contemplated lyophilized composition may be reconstituted in about 30 to about 360 seconds, about 30 to about 300 seconds, about 30 to about 150 seconds, about 30 to about 120 seconds, about 40 to about 90 seconds, or about 60 to about 150 seconds.

In another aspect, the invention provides a pharmaceutically acceptable formulation for parenteral administration, prepared by a process comprising: a) providing a composition comprising a plurality of therapeutic particles each comprising a copolymer having a hydrophobic polymer segment and a hydrophilic polymer segment; and an active agent; b) adding a sugar alcohol and a cyclodextrin to said composition; c) lyophilizing the composition to form a lyophilized composition; d) reconstituting the lyophilized composition to form the formulation suitable for parenteral administration. In some embodiments, such reconstituting can advantageously be managed with simple manual mixing for a few minutes. The reconstituted product attributes (e.g., drug purity and/or release profile) may be substantially unchanged from a pre-lyophilized composition (e.g., suspension).

In some embodiments, a contemplated lyophilization process may be completed more quickly as compared to a lyophilization process that does not contain a sugar alcohol (e.g., mannitol) and/or a cyclodextrin. For example, in certain embodiments, a contemplated lyophilization process may comprise a cycle lasting fewer than 72 hours. For instance, a contemplated lyophilization process cycle may be about 12 to about 72 hours long, about 24 to about 72 hours long, about 36 to about 72 hours long, or about 48 to about 72 hours long. In certain embodiments, a contemplated lyophilization process cycle may be about 60 hours long.

In some embodiments, a contemplated lyophilization cycle may comprise a series of stages that may differ by, e.g., length of time, temperature, and/or pressure. In certain embodiments, a contemplated lyophilization cycle may comprise the following stages: (1) shelf load and freezing, (2) primary dry, and (3) secondary dry and storage.

In some embodiments, the shelf load and freezing stage may be used to conduct an annealing procedure. Without wishing to be bound by any theory, it is believed that an annealing procedure, carried out by performing a temperature cycling, alters the physical and/or chemical properties of a material. For the contemplated formulations, annealing may be used, in some embodiments, to prepare a frozen nanoparticle suspension comprising a substantially crystalline sugar alcohol. Without wishing to be bound by any theory, it is believed that a substantially crystalline sugar alcohol facilitates moisture removal by creating more open channels during drying of the frozen nanoparticle suspension. Furthermore, in some embodiments, an annealing procedure can be used to alter a critical temperature of a material (e.g., a sugar alcohol) such that the material does not undergo a glass transition in a particular temperature range during lyophilization.

Glass transitions may be observed, e.g., using differential scanning calorimetry. An annealing process may then be designed to transition a material (e.g., a sugar alcohol) to a particular solid state. For example, a nanoparticle suspension in a liquid state may be frozen at a first temperature that is below the glass transition temperature of the sugar alcohol and then annealed by raising the temperature of the frozen nanoparticle suspension to a second temperature that is between that of the glass transition temperature and the solid/liquid transition temperature of the frozen nanoparticle suspension. For example, the first temperature may be least about 5° C., at least about 10° C., at least about 15° C., or at least about 20° C. less than the glass transition temperature of the sugar alcohol. In certain embodiments, the second temperature may be at least about 5° C., at least about 10° C., at least about 15° C., or at least about 20° C. greater than the glass transition temperature of the sugar alcohol. In some embodiments, following the second, elevated temperature, the temperature of the frozen nanoparticle suspension may be decreased, e.g., to the original first temperature or any other suitable temperature. The frozen nanoparticle suspension may be independently held at the first and/or the second temperature and/or the third temperature for a period of time, e.g., between about 1 hour and about 10 hours, between 1 hour and about 5 hours, between about 1 hour and about 4 hours, or between about 2 hours and about 5 hours. In one embodiment, the frozen nanoparticle suspension may be held at the first temperature for about 2 hours, held at the second temperature for about 3 hours, and held at the third temperature for about 2 hours.

Following the freezing stage, the lyophilization cycle proceeds to the primary drying stage, where the frozen nanoparticle suspension is subjected to a vacuum to facilitate sublimation. The temperature of the shelf on which the frozen nanoparticle suspensions are held and the vacuum pressure may be selected such that the frozen nanoparticle suspension maintains a desired temperature during the initial sublimation phase of the primary drying stage. For example, in some embodiments, it may be desirable to select a shelf temperature and vacuum pressure such that the temperature of the frozen nanoparticle suspension is below the collapse temperature (i.e., the temperature at which a lyophilization cake begins losing its structure, as determined by, e.g., freeze dry microscopy). In some embodiments, the temperature of the frozen nanoparticle suspension during the drying stage may be at least about 1° C., at least about 2° C., at least about 3° C., at least about 4° C., or at least about 5° C. less than the collapse temperature. In some embodiments, the vacuum pressure during the primary drying stage may be between about 100 mTorr and about 500 mTorr, between about 150 mTorr and about 400 mTorr, or between about 200 mTorr and about 300 mTorr. In one embodiment, the vacuum pressure during the primary drying stage may be about 250 mTorr. In some embodiments, the length of the primary drying stage may be between about 12 hours and about 96 hours, between about 12 hours and about 72 hours, or between about 24 hours and about 48 hours. In one embodiment, the drying time may be about 1.5 days.

In some embodiments, a secondary drying and storage stage follows the freezing stage and results in formation of the lyophilized composition. In certain embodiments, about 70% to about 90% by weight of the water in the frozen nanoparticle suspension has been removed prior to commencement of the secondary drying stage. In some embodiments, the secondary drying may be conducted at a higher temperature than the primary drying. For example, in some embodiments, the secondary drying temperature may be between about 0° C. and about 40° C., between about 10° C. and about 40° C., between about 20° C. and about 40° C., or between about 30° C. and about 40° C. In one embodiment, the secondary drying temperature may be about 35° C. In certain embodiments, the secondary drying may be carried out under vacuum, e.g., at about the same pressure as the primary drying.

Upon completion of the secondary drying, the lyophilized composition may be stored under vacuum until being removed from the lyophilizer. In some embodiments, the lyophilized composition may be stored at a lower temperature than the secondary drying. In some embodiments, the lyophilized composition may be stored at between about 0° C. and about 30° C., or between about 10° C. and about 30° C. In one embodiment, the lyophilized composition may be stored at about 20° C. under a vacuum of about 250 mTorr.

In yet another aspect, the invention provides a pharmaceutically acceptable formulation for parenteral administration, prepared by a process comprising: a) providing a composition comprising a plurality of therapeutic particles each comprising a copolymer having a hydrophobic polymer segment and a hydrophilic polymer segment; and an active agent; b) adding a disaccharide and a cyclodextrin to said composition; c) lyophilizing the composition to form a lyophilized composition; d) reconstituting the lyophilized composition to form the formulation suitable for parenteral administration. In some embodiments, such reconstituting can advantageously be managed with simple manual mixing for a few minutes. The reconstituted product attributes (e.g. drug purity and/or release profile) may be substantially unchanged from a pre-lyophilized composition (e.g. suspension).

The step of lyophilizing may comprise freezing the composition at a temperature of greater than about −40° C., or e.g. less than about −30° C., forming a frozen composition; and drying the frozen composition to form the lyophilized composition. The step of drying may occur at about 50 mTorr at a temperature of about −25 to about −34° C., or about −30 to about −34° C.

In another aspect, the invention provides a method of preventing substantial aggregation of particles in a pharmaceutical nanoparticle composition comprising adding a sugar alcohol and a salt to the lyophilized formulation to prevent aggregation of the nanoparticles upon reconstitution. In an embodiment, a cyclodextrin is also added to the lyophilized formulation. In yet another aspect, the invention provides a method of preventing substantial aggregation of particles in a pharmaceutical nanoparticle composition comprising adding a sugar alcohol and a cyclodextrin to the lyophilized formulation to prevent aggregation of the nanoparticles upon reconstitution.

Nanoparticles disclosed herein may be combined with pharmaceutically acceptable carriers to form a pharmaceutical composition, according to one aspect. As would be appreciated by one of skill in this art, the carriers may be chosen based on the route of administration as described below, the location of the target issue, the drug being delivered, the time course of delivery of the drug, etc.

The pharmaceutical compositions of this invention can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles of the present invention are administered to a subject in need thereof systemically, e.g., parenterally, or by intravenous infusion or injection.

In some embodiments, a contemplated lyophilized pharmaceutical composition may be suspended in a container using about 6 mL to about 7 mL, or about 6.6 mL, of solution. In some embodiments, a dose of a contemplated lyophilized pharmaceutical composition may comprise about 25 mg to about 50 mg of active agent, about 25 mg to about 40 mg of active agent, about 30 mg to about 40 mg of active agent, about 30 mg to about 37 mg of active agent, or about 33 mg of active agent

### Nanoparticles

In general, a “nanoparticle” refers to any particle having a diameter of less than 1000 nm, e.g., about 10 nm to about 200 nm. Disclosed therapeutic nanoparticles may include nanoparticles having a diameter of about 60 to about 200 nm, about 60 to about 190 nm, or about 70 to about 190 nm, or about 60 to about 180 nm, or about 70 nm to about 180 nm, or about 50 nm to about 200 nm, or about 60 to about 120 nm, or about 70 to about 120 nm, or about 80 to about 120 nm, or about 90 to about 120 nm, or about 100 to about 120 nm, or about 60 to about 130 nm, or about 70 to about 130 nm, or about 80 to about 130 nm, or about 90 to about 130 nm, or about 100 to about 130 nm, or about 110 to about 130 nm, or about 60 to about 140 nm, or about 70 to about 140 nm, or about 80 to about 140 nm, or about 90 to about 140 nm, or about 100 to about 140 nm, or about 110 to about 140 nm, or about 60 to about 150 nm, or about 70 to about 150 nm, or about 80 to about 150 nm, or about 90 to about 150 nm, or about 100 to about 150 nm, or about 110 to about 150 nm, or about 120 to about 150 nm.

Nanoparticles disclosed herein include one, two, three or more biocompatible and/or biodegradable polymers. For example, a contemplated nanoparticle may include about 35 to about 99.6 weight percent, in some embodiments about 50 to about 99.6 weight percent, in some embodiments about 50 to about 99.5 weight percent, in some embodiments about 50 to about 99 weight percent, in some embodiments about 50 to about 98 weight percent, in some embodiments about 50 to about 97 weight percent, in some embodiments about 50 to about 96 weight percent, in some embodiments about 50 to about 95 weight percent, in some embodiments about 50 to about 94 weight percent, in some embodiments about 50 to about 93 weight percent, in some embodiments about 50 to about 92 weight percent, in some embodiments about 50 to about 91 weight percent, in some embodiments about 50 to about 90 weight percent, in some embodiments about 50 to about 85 weight percent, and in some embodiments about 50 to about 80 weight percent of one or more block copolymers that include a biodegradable polymer and poly(ethylene glycol) (PEG), and about 0 to about 50 weight percent of a biodegradable homopolymer.

In some embodiments, disclosed nanoparticles may include about 0.2 to about 35 weight percent, about 0.2 to about 30 weight percent, about 0.2 to about 20 weight percent, about 0.2 to about 10 weight percent, about 0.2 to about 5 weight percent, about 0.5 to about 5 weight percent, about 0.75 to about 5 weight percent, about 1 to about 5 weight percent, about 2 to about 5 weight percent, about 3 to about 5 weight percent, about 1 to about 30 weight percent, about 1 to about 20 weight percent, about 2 to about 20 weight percent, about 5 to about 20 weight percent, about 1 to about 15 weight percent, about 2 to about 15 weight percent, about 3 to about 15 weight percent, about 4 to about 15 weight percent, about 5 to about 15 weight percent, about 1 to about 10 weight percent, about 2 to about 10 weight percent, about 3 to about 10 weight percent, about 4 to about 10 weight percent, about 5 to about 10 weight percent, about 10 to about 30 weight percent, or about 15 to about 25 weight percent of an active agent.

In one set of embodiments, the nanoparticles can have an interior and a surface, where the surface has a composition different from the interior, i.e., there may be at least one compound present in the interior but not present on the surface (or vice versa), and/or at least one compound is present in the interior and on the surface at differing concentrations. For example, in one embodiment, a compound, such as a targeting moiety, may be present in both the interior and the surface of the particle, but at a higher concentration on the surface than in the interior of the particle. Although in some cases, the concentration in the interior of the particle may be essentially nonzero, i.e., there is a detectable amount of the compound present in the interior of the particle.

In some cases, the interior of the particle is more hydrophobic than the surface of the particle. For instance, the interior of the particle may be relatively hydrophobic with respect to the surface of the particle, and a drug or other payload may be hydrophobic, and readily associates with the relatively hydrophobic center of the particle. The drug or other payload can thus be contained within the interior of the particle, which can shelter it from the external environment surrounding the particle (or vice versa). For instance, a drug or other payload contained within a particle administered to a subject will be protected from a subject's body, and the body may also be substantially isolated from the drug for at least a period of time.

For example, disclosed herein is a therapeutic polymeric nanoparticle comprising a first non-functionalized polymer; an optional second non-functionalized polymer; an optional functionalized polymer comprising a targeting moiety; and a therapeutic agent. In a particular embodiment, the first non-functionalized polymer is PLA, PLGA, or PEG, or copolymers thereof, e.g., a diblock co-polymer PLA-PEG. For example, exemplary nanoparticles may have a PEG corona with a density of about 0.065 g/cm³, or about 0.01 to about 0.10 g/cm³.

Disclosed nanoparticles may be stable (e.g., retain substantially all active agent) for example in a solution that may contain a sugar alcohol and a cyclodextrin, for at least about 3 days, about 4 days or at least about 5 days at room temperature, or at 25° C.

In some embodiments, a contemplated nanoparticle may comprise a cyclodextrin. A suitable cyclodextrin may include α-cyclodextrin, β-cyclodextrin, γ-cyclodextrin, or mixtures thereof. Exemplary cyclodextrins contemplated for use in the nanoparticles disclosed herein include hydroxypropyl-β-cyclodextrin (HPbCD), hydroxyethyl-β-cyclodextrin, sulfobutylether-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-β-cyclodextrin, glucosyl-β-cyclodextrin, and maltosyl-β-cyclodextrin. In some embodiments, the cyclodextrin may be covalently attached to polymer. For example, in some embodiments, the cyclodextrin may be covalently attached to chitosan.

For example, in some embodiments, a contemplated nanoparticle may comprise about 0.05 to about 35 weight percent of a cyclodextrin, in some embodiments about 0.05 to about 30 weight percent of a cyclodextrin, in some embodiments about 0.1 to about 30 weight percent of a cyclodextrin, in some embodiments about 0.5 to about 30 weight percent of a cyclodextrin, in some embodiments about 1 to about 30 weight percent of a cyclodextrin, in some embodiments about 2 to about 30 weight percent of a cyclodextrin, in some embodiments about 5 to about 30 weight percent of a cyclodextrin, in some embodiments about 10 to about 30 weight percent of a cyclodextrin, in some embodiments about 15 to about 30 weight percent of a cyclodextrin, in some embodiments about 20 to about 30 weight percent of a cyclodextrin, in some embodiments about 15 to about 25 weight percent of a cyclodextrin, in some embodiments about 5 to about 25 weight percent of a cyclodextrin, in some embodiments about 5 to about 20 weight percent of a cyclodextrin, or in some embodiments about 5 to about 15 weight percent of a cyclodextrin.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

Nanoparticles may have controlled release properties, e.g., may be capable of delivering an amount of a therapeutic agent to a patient, e.g., to specific site in a patient, over an extended period of time, e.g., over 1 day, 1 week, or more.

In some embodiments, disclosed nanoparticles substantially immediately release (e.g., over about 1 minute to about 30 minutes) less than about 2%, less than about 4%, less than about 5%, or less than about 10% of an active agent, for example when placed in a phosphate buffer solution at room temperature and/or at 37° C.

In another embodiment, a disclosed nanoparticle may release less than about 40%, less than 50%, or less than 60%, less than 70% of an active agent for example when placed in a phosphate buffer solution at room temperature or at 37° C., for 0.5 hour or more. In one embodiment, a disclosed nanoparticle may release less than about 70% of the therapeutic agent over 0.5 hour when placed in a phosphate buffer solution at 37° C.

In another embodiment, a disclosed nanoparticle may release less than about 20%, less than about 30%, less than about 40%, less than 50%, or even less than 60% (or more) for example when placed in a phosphate buffer solution at room temperature or at 37° C., for 1 day or more. In one embodiment, a disclosed nanoparticle may release less than about 60% of the therapeutic agent over 2 hours when placed in a phosphate buffer solution at room temperature.

In some embodiments, after administration to a subject or patient of a disclosed nanoparticle or a composition that includes a disclosed nanoparticle, the peak plasma concentration (Cₘₐₓ) of the therapeutic agent in the patient is substantially higher as compared to a Cₘₐₓ of the therapeutic agent if administered alone (e.g., not as part of a nanoparticle).

In another embodiment, a disclosed nanoparticle including a therapeutic agent, when administered to a subject, may have a tₘₐₓ of therapeutic agent substantially longer as compared to a tₘₐₓ of the therapeutic agent administered alone.

Libraries of such particles may also be formed. For example, by varying the ratios of the two (or more) polymers within the particle, these libraries can be useful for screening tests, high-throughput assays, or the like. Entities within the library may vary by properties such as those described above, and in some cases, more than one property of the particles may be varied within the library. Accordingly, one embodiment is directed to a library of nanoparticles having different ratios of polymers with differing properties. The library may include any suitable ratio(s) of the polymers.

In some embodiments, the biocompatible polymer is a hydrophobic polymer. Non-limiting examples of biocompatible polymers include polylactide, polyglycolide, and/or poly(lactide-co-glycolide).

### Polymers

In some embodiments, the nanoparticles of the invention comprise a matrix of polymers and a therapeutic agent. In some embodiments, a therapeutic agent and/or targeting moiety (i.e., a low-molecular weight PSMA ligand) can be associated with at least part of the polymeric matrix. For example, in some embodiments, a targeting moiety (e.g., ligand) can be covalently associated with the surface of a polymeric matrix. In some embodiments, covalent association is mediated by a linker. The therapeutic agent can be associated with the surface of, encapsulated within, surrounded by, and/or dispersed throughout the polymeric matrix.

A wide variety of polymers and methods for forming particles therefrom are known in the art of drug delivery. In some embodiments, the disclosure is directed toward nanoparticles with at least two macromolecules, wherein the first macromolecule comprises a first polymer bound to a low-molecular weight ligand (e.g., targeting moiety); and the second macromolecule comprising a second polymer that is not bound to a targeting moiety. The nanoparticle can optionally include one or more additional, unfunctionalized, polymers.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeating units forming the copolymer may be arranged in any fashion. For example, the repeating units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and ether bonds (e.g., R—O—R′ bonds). In some embodiments of the invention, a biodegradable polymer, such as a hydrolyzable polymer, containing carboxylic acid groups, may be conjugated with poly(ethylene glycol) repeat units to form a poly(ester-ether). A polymer (e.g., copolymer, e.g., block copolymer) containing poly(ethylene glycol) repeat units can also be referred to as a “PEGylated” polymer.

It is contemplated that PEG may be terminated and include an end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like

A disclosed particle can for example comprise a diblock copolymer of PEG and PL(G)A, wherein for example, the PEG portion may have a number average molecular weight of about 1,000-20,000, e.g., about 2,000-20,000, e.g., about 2 to about 10,000, and the PL(G)A portion may have a number average molecular weight of about 5,000 to about 20,000, or about 5,000-100,000, e.g., about 20,000-70,000, e.g., about 15,000-50,000.

For example, disclosed here is an exemplary therapeutic nanoparticle that includes about 10 to about 99 weight percent poly(lactic) acid-poly(ethylene)glycol copolymer or poly(lactic)-co-poly (glycolic) acid-poly(ethylene)glycol copolymer, or about 20 to about 80 weight percent, about 40 to about 80 weight percent, or about 30 to about 50 weight percent, or about 70 to about 90 weight percent poly(lactic) acid-poly(ethylene)glycol copolymer or poly(lactic)-co-poly (glycolic) acid-poly(ethylene)glycol copolymer. Exemplary poly(lactic) acid-poly(ethylene)glycol copolymers can include a number average molecular weight of about 15 to about 20 kDa, or about 10 to about 25 kDa of poly(lactic) acid and a number average molecular weight of about 4 to about 6, or about 2 kDa to about 10 kDa of poly(ethylene)glycol.

Disclosed nanoparticles may optionally include about 1 to about 50 weight percent poly(lactic) acid or poly(lactic) acid-co-poly (glycolic) acid (which does not include PEG), or may optionally include about 1 to about 50 weight percent, or about 10 to about 50 weight percent or about 30 to about 50 weight percent poly(lactic) acid or poly(lactic) acid-co-poly (glycolic) acid. For example, poly(lactic) or poly(lactic)-co-poly(glycolic) acid may have a number average molecule weight of about 5 to about 15 kDa, or about 5 to about 12 kDa. Exemplary PLA may have a number average molecular weight of about 5 to about 10 kDa. Exemplary PLGA may have a number average molecular weight of about 8 to about 12 kDa.

### Targeting Moieties

Provided herein are nanoparticles that may include an optional targeting moiety, i.e., a moiety able to bind to or otherwise associate with a biological entity, for example, a membrane component, a cell surface receptor, prostate specific membrane antigen, or the like. A targeting moiety present on the surface of the particle may allow the particle to become localized at a particular targeting site, for instance, a tumor, a disease site, a tissue, an organ, a type of cell, etc. As such, the nanoparticle may then be “target specific.” The drug or other payload may then, in some cases, be released from the particle and allowed to interact locally with the particular targeting site.

In a particular embodiment, the drug or other payload may be released in a controlled release manner from the particle and allowed to interact locally with the particular targeting site (e.g., a tumor). The term “controlled release” (and variants of that term) as used herein (e.g., in the context of “controlled-release system”) is generally meant to encompass release of a substance (e.g., a drug) at a selected site or otherwise controllable in rate, interval, and/or amount. Controlled release encompasses, but is not necessarily limited to, substantially continuous delivery, patterned delivery (e.g., intermittent delivery over a period of time that is interrupted by regular or irregular time intervals), and delivery of a bolus of a selected substance (e.g., as a predetermined, discrete amount if a substance over a relatively short period of time (e.g., a few seconds or minutes)).

In one embodiment, a disclosed nanoparticle includes a targeting moiety that is a low-molecular weight ligand, e.g., a low-molecular weight PSMA ligand.

For example, the low-molecular weight PSMA ligand may be:

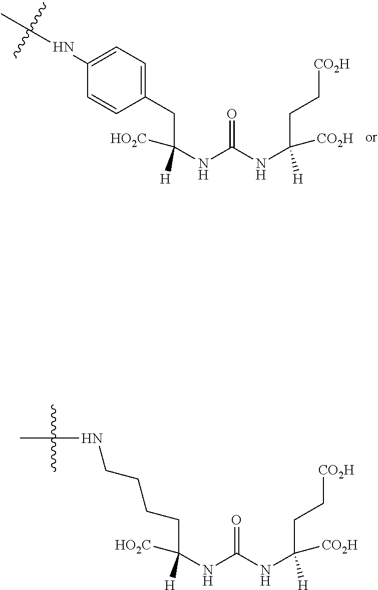 [SMILES: *Nc1ccc(C[C@H](NC(=O)N[C@@H](CCC(=O)O)C(=O)O)C(=O)O)cc1 ; "or" ; *NCCCC[C@H](NC(=O)N[C@@H](CCC(=O)O)C(=O)O)C(=O)O]

and enantiomers, stereoisomers, rotamers, tautomers, diastereomers, or racemates thereof. Particularly, the butyl-amine compound has the advantage of ease of synthesis, especially because of its lack of a benzene ring.

For example, a disclosed nanoparticle may include a conjugate represented by:

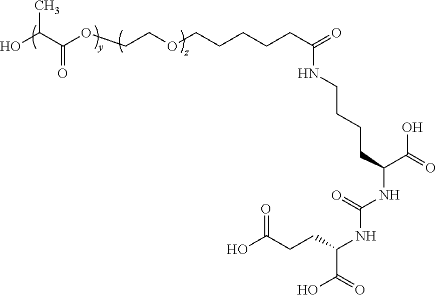

where y is about 222 and z is about 114.

For example, a disclosed nanoparticle includes a polymeric compound selected from:

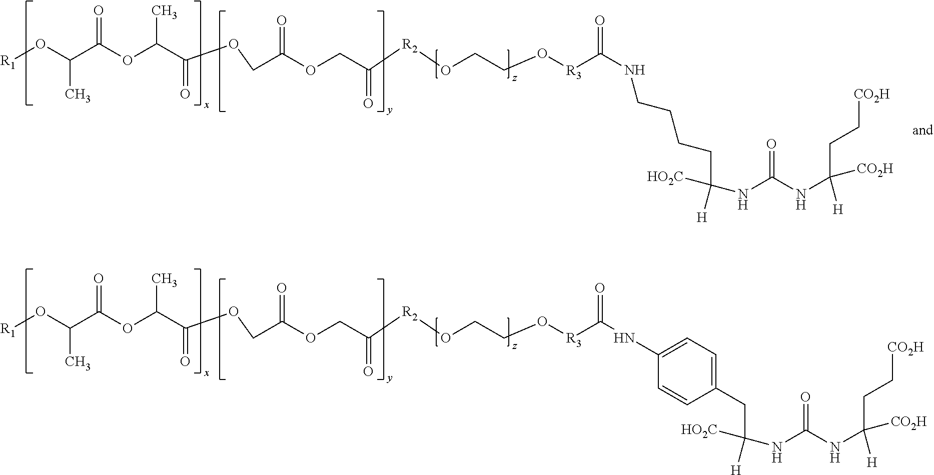

wherein R₁ is selected from the group consisting of H, and a C₁-C₂₀ alkyl group optionally substituted with halogen;

R₂ is a bond, an ester linkage, or amide linkage;

R₃ is an C₁-C₁₀ alkylene or a bond;

x is 50 to about 1500, for example about 170 to about 260;

y is 0 to about 50, for example y is 0; and

z is about 30 to about 456, or about 30 to about 200, for example, z is about 80 to about 130.

### Therapeutic Agents

In an embodiment, an active or therapeutic agent may (or may not be) conjugated to e.g. a disclosed polymer that forms part of a disclosed nanoparticle, e.g. an active agent may be conjugated (e.g. covalently bound, e.g. directly or through a linking moiety) to PLA or PGLA, or a PLA or PLGA portion of a copolymer such as PLA-PEG or PLGA-PEG.

In one aspect, any agent including, for example, therapeutic agents (e.g. anti-cancer agents or anti-inflammatory agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be delivered by the disclosed nanoparticles. Exemplary agents to be delivered in accordance with the present invention include, but are not limited to, small molecules (e.g., cytotoxic agents or anti-inflammatory agents), nucleic acids (e.g., siRNA, RNAi, and mircoRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of cancer.

In a particular embodiment, the drug or other payload may be released in a controlled release manner from the particle and allowed to interact locally with the particular targeting site (e.g., a tumor or inflamed tissue). The term “controlled release” (and variants of that term) as used herein (e.g., in the context of “controlled-release system”) is generally meant to encompass release of a substance (e.g., a drug) at a selected site or otherwise controllable in rate, interval, and/or amount. Controlled release encompasses, but is not necessarily limited to, substantially continuous delivery, patterned delivery (e.g., intermittent delivery over a period of time that is interrupted by regular or irregular time intervals), and delivery of a bolus of a selected substance (e.g., as a predetermined, discrete amount if a substance over a relatively short period of time (e.g., a few seconds or minutes)).

The active agent or drug may be a therapeutic agent (e.g. a chemotherapeutic) such as mTor inhibitors (e.g., sirolimus, temsirolimus, or everolimus), vinca alkaloids (e.g. vinorelbine or vincristine), a diterpene derivative, a taxane (e.g. paclitaxel or its derivatives such as DHA-paclitaxel or PG-paclitaxel, or docetaxel), a cardiovascular agent (e.g. a diuretic, a vasodilator, angiotensin converting enzyme, a beta blocker, an aldosterone antagonist, or a blood thinner), a corticosteroid, an antimetabolite or antifolate agent (e.g. methotrexate), a chemotherapeutic agent (e.g. epothilone B), an alkylating agent (e.g. bendamustine), or the active agent or drug may be an siRNA.

In one set of embodiments, the payload is a drug or a combination of more than one drug. Such particles may be useful, for example, in embodiments where a targeting moiety may be used to direct a particle containing a drug to a particular localized location within a subject, e.g., to allow localized delivery of the drug to occur. Exemplary therapeutic agents include chemotherapeutic agents such as doxorubicin (Adriamycin), gemcitabine (Gemzar), daunorubicin, procarbazine, mitomycin, cytarabine, etoposide, methotrexate, venorelbine, 5-fluorouracil (5-FU), vinca alkaloids such as vinblastine or vincristine; bleomycin, paclitaxel (taxol), docetaxel (taxotere), cabazitaxel, aldesleukin, asparaginase, busulfan, carboplatin, cladribine, camptothecin, CPT-11, 10-hydroxy-7-ethylcamptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloroadenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, and combinations thereof.

Non-limiting examples of potentially suitable drugs include anti-cancer agents, including, for example, cabazitaxel, mitoxantrone, and mitoxantrone hydrochloride. In another embodiment, the payload may be an anti-cancer drug such as 20-epi-1, 25 dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, cabazitaxel, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinarnide, RNAi, rogletimide, rohitukine, romurtide, roquinimex, rubiginone B1, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

Non-limiting examples of potentially suitable drugs also include anti-inflammatory agents, including, for example, anti-inflammatory steroids and non-steroidal anti-inflammatory agents (NSAIDs). Non-limiting examples of anti-inflammatory agents include methotrexate, cyclosporine, alclometasone, azathioprine, beclometasone dipropionate, betamethasone dipropionate, budesonide, celecoxib, chloroprednisone, ciclesonide, cortisol, cortisporin, cortivazol, deflazacort, dexamethasone, fludroxycortide, flunisolide, fluocinonide, fluocortolone, fluorometholone, fluticasone, fluticasone furoate, fluticasone propionate, glucocorticoids, hydrocortamate, megestrol acetate, mesalazine, meprednisone, 6-mercaptopurine, methylprednisolone, mometasone furoate, paramethasone, prednisolone, prednisone, prednylidene, pregnadiene, pregnatriene, pregnene, proctosedyl, rimexolone, tetrahydrocorticosterone, tobramycin/dexamethasone, triamcinolone, and ulobetasol.

### EXAMPLES

The invention now being generally described, it will be more readily understood by reference to the following examples which are included merely for purposes of illustration of certain aspects and embodiments of the present invention, and are not intended to limit the invention in any way.

### Example 1: Exemplary Nanoparticle Preparation—Emulsion Process

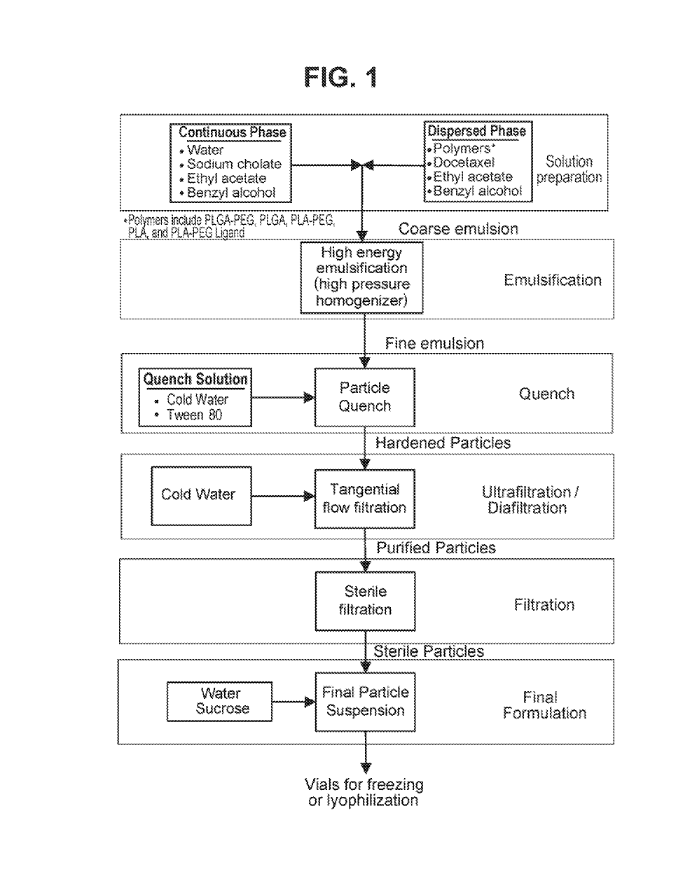

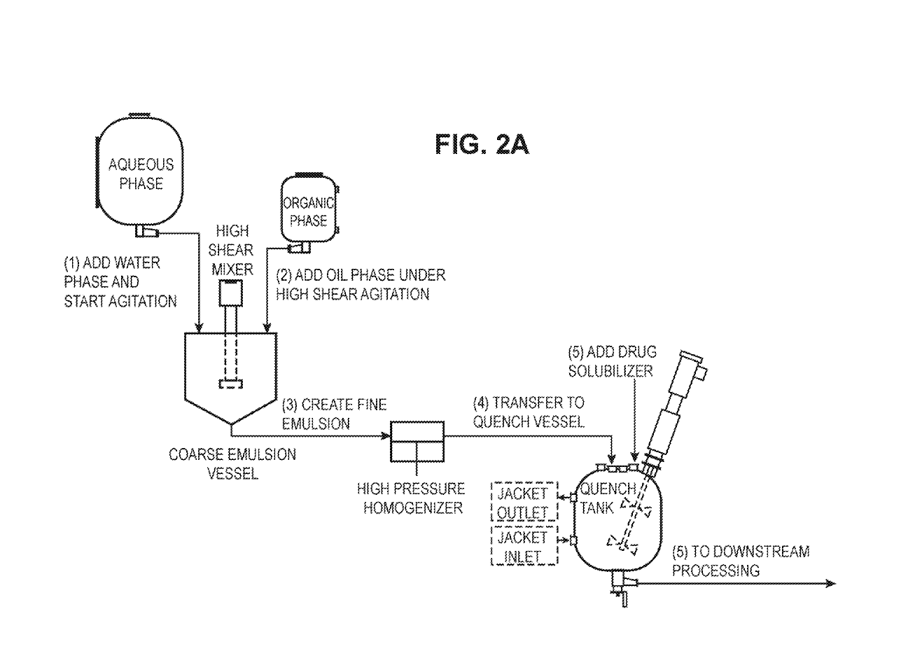

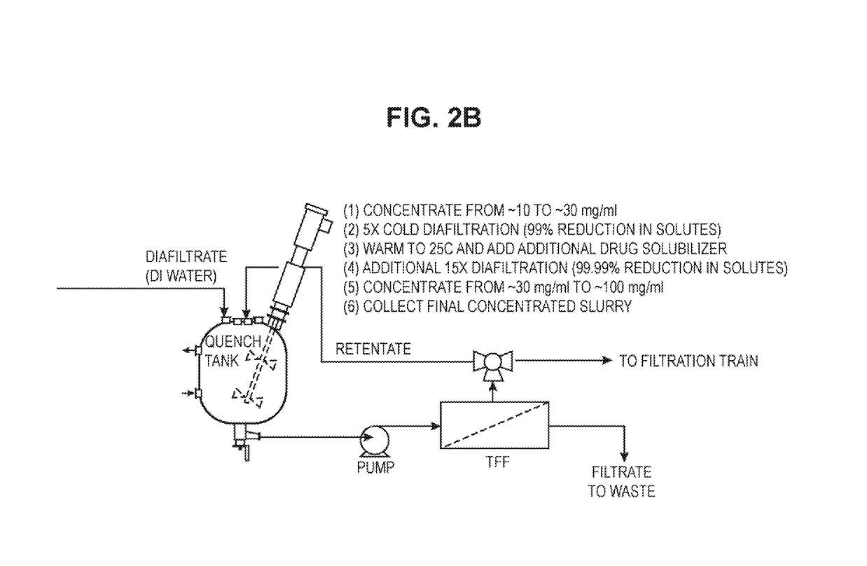

An exemplary process for preparing contemplated nanoparticles is illustrated in FIGS. 1, 2A, and 2B.

An organic phase is formed composed of a mixture of docetaxel (DTXL) and polymer (homopolymer, co-polymer, and co-polymer with ligand). The organic phase is mixed with an aqueous phase at approximately a 1:2 ratio (oil phase:aqueous phase) where the aqueous phase is composed of a surfactant (0.25% sodium cholate) and some dissolved solvent (4% ethyl acetate, 2% benzyl alcohol). In order to achieve high drug loading, about 30% solids in the organic phase is used.

The primary, coarse emulsion is formed by the combination of the two phases under simple mixing or through the use of a rotor stator homogenizer. The rotor/stator yields a homogeneous milky solution, while the stir bar produces a visibly larger coarse emulsion. It is observed that the stir bar method results in significant oil phase droplets adhering to the side of the feed vessel, suggesting that while the coarse emulsion size is not a process parameter critical to quality, it should be made suitably fine in order to prevent yield loss or phase separation. Therefore the rotor stator is used as the standard method of coarse emulsion formation, although a high speed mixer may be suitable at a larger scale.

The primary emulsion is then formed into a fine emulsion through the use of a high pressure homogenizer. The size of the coarse emulsion does not significantly affect the particle size after successive passes (103) through the homogenizer.

After 2-3 passes the particle size is not significantly reduced, and successive passes can even cause a particle size increase. The organic phase is emulsified 5:1 O:W with standard aqueous phase, and multiple discreet passes are performed, quenching a small portion of emulsion after each pass. The indicated scale represents the total solids of the formulation.

The effect of scale on particle size shows scale dependence. The trend shows that in the 2-10 g batch size range, larger batches produce smaller particles. It has been demonstrated that this scale dependence is eliminated when considering greater than 10 g scale batches. The amount of solids used in the oil phase is about 30%.

Table A summarizes the emulsification process parameters.

TABLE A

| Parameter | Value |
|---|---|
| Coarse emulsion formation | High shear mixer |
| Homogenizer feed pressure | 2500 psi per chamber |
| Interaction chamber(s) | 4 × 200 μm Z-chamber |
| Number of homogenizer passes | 1 pass |
| Water phase [sodium cholate] | 0.25-0.35% |
| W:O ratio | 2:1 |
| [Solids] in oil phase | 30% |

The fine emulsion is then quenched by addition to deionized water at a given temperature under mixing. In the quench unit operation, the emulsion is added to a cold aqueous quench under agitation. This serves to extract a significant portion of the oil phase solvents, effectively hardening the nanoparticles for downstream filtration. Chilling the quench significantly improves drug encapsulation. The quench:emulsion ratio is approximately 5:1.

A solution of 35% (wt %) of Tween 80 is added to the quench to achieve approximately 4% Tween 80 overall. After the emulsion is quenched a solution of Tween-80 is added which acts as a drug solubilizer, allowing for effective removal of unencapsulated drug during filtration. Table B indicates each of the quench process parameters.

TABLE B
Summary quench process parameters.

| Parameter | Value |
|---|---|
| Initial quench temperature | <5° C. |
| [Tween-80] solution | 35% |
| Tween-80:drug ratio | 25:1 |
| Q:E ratio | 10:1 |
| Quench hold/processing temp | ≤5° C. (with current |
| | 5:1 Q:E ratio, 25:1 |
| | Tween-80:drug ratio) |

The temperature must remain cold enough with a dilute enough suspension (low enough concentration of solvents) to remain below the T_{g} of the particles. If the Q:E ratio is not high enough, then the higher concentration of solvent plasticizes the particles and allows for drug leakage. Conversely, colder temperatures allow for high drug encapsulation at low Q:E ratios (to ˜3:1), making it possible to run the process more efficiently.

The nanoparticles are then isolated through a tangential flow filtration process to concentrate the nanoparticle suspension and buffer exchange the solvents, free drug, and drug solubilizer from the quench solution into water. A regenerated cellulose membrane is used with a molecular weight cutoffs (MWCO) of 300.

A constant volume diafiltration (DF) is performed to remove the quench solvents, free drug and Tween-80. To perform a constant-volume DF, buffer is added to the retentate vessel at the same rate the filtrate is removed. The process parameters for the TFF operations are summarized in Table C. Crossflow rate refers to the rate of the solution flow through the feed channels and across the membrane. This flow provides the force to sweep away molecules that can foul the membrane and restrict filtrate flow. The transmembrane pressure is the force that drives the permeable molecules through the membrane.

TABLE C
TFF Parameters

| Parameter | Optimized Value | |
|---|---|---|
| Membrane Material | Regenerated cellulose - Coarse | |
| | Screen Membrane | |
| Molecular Weight Cut off | 300 | kDa |
| Crossflow Rate | 3.7-10 | L/min/m² |
| Transmembrane Pressure | ~5 | psid |
| Concentration of Nanoparticle | 30-50 | mg/ml |
| Suspension for Diafiltration | | |
| Number of Diavolumes | 20) | |
| Membrane Area | 5 | m²/kg |

The filtered nanoparticle slurry is then thermal cycled to an elevated temperature during workup. A small portion (typically 5-10%) of the encapsulated drug is released from the nanoparticles very quickly after its first exposure to 25° C. Because of this phenomenon, batches that are held cold during the entire workup are susceptible to free drug or drug crystals forming during delivery or any portion of unfrozen storage. By exposing the nanoparticle slurry to elevated temperature during workup, this ‘loosely encapsulated’ drug can be removed and improve the product stability at the expense of a small drop in drug loading. Table D summarizes two examples of 25° C. processing. Other experiments have shown that the product is stable enough after ˜2-4 diavolumes to expose it to 25° C. without losing the majority of the encapsulated drug. 5 diavolumes is used as the amount for cold processing prior to the 25° C. treatment.

TABLE D

| | | Lots A | | Lots B | |
|---|---|---|---|---|---|
| Drug load | Cold workup | 11.3% | | 9.7% | |
| | 25° C. workup¹ | 8.7-9.1% | | 9.0-9.9% | |
| Stability² | Cold workup | <1 | day | <1 | day |
| | 25° C. workup¹ | 5-7 | days | 2-7 | days |

| | | | |
|---|---|---|---|
| In vitro burst³ | Cold workup | ~10% | Not |
| | 25° C. workup¹ | ~2% | performed |

¹25° C. workup sublots were exposed to 25° C. after at least 5 diavolumes for various periods of time. Ranges are reported because there were multiple sublots with 25° C. exposure.
²Stability data represents the time that final product could be held at 25° C. at 10-50 mg/ml nanoparticle concentrations prior to crystals forming in the slurry (visible by microscopy)
³In vitro burst represents the drug released at the first time point (essentially immediately)

After the filtration process, the nanoparticle suspension is passed through a sterilizing grade filter (0.2 μm absolute). Pre-filters are used to protect the sterilizing grade filter in order to use a reasonable filtration area/time for the process. Values are as summarized in Table E.

TABLE E

| Parameter | O Value | | Effect |
|---|---|---|---|
| Nanoparticle | 50 | mg/ml | Yield losses are higher at |
| Suspension | | | higher [NP], but the ability |
| Concentration | | | to filter at 50 mg/ml obviates |
| | | | the need to aseptically |
| | | | concentrate after filtration |
| Filtration flow | ~1.3 | L/min/m² | Filterability decreases as |
| rate | | | flow rate increases |

The pre-filter has Seitz PDD1 depth filter media in Pall SUPRAcap or Stax filter cartridges. 0.2 m² of filtration surface area per kg of nanoparticles for depth filters and 1.3 m² of filtration surface area per kg of nanoparticles for the sterilizing grade filters can be used.

### Example 2: Lyophilized Composition with Mannitol and Hydroxypropyl-β-Cyclodextrin (HPbCD)

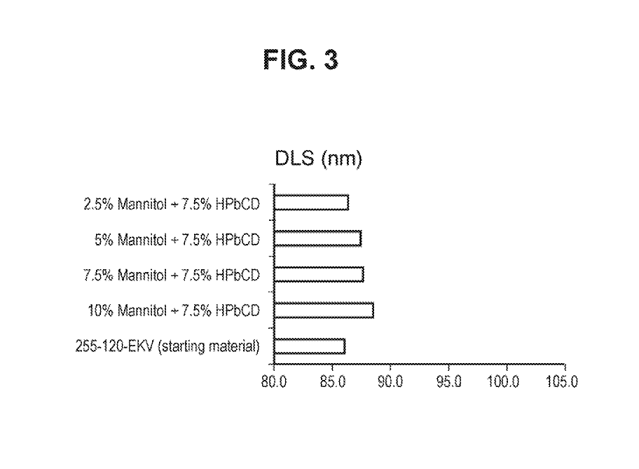

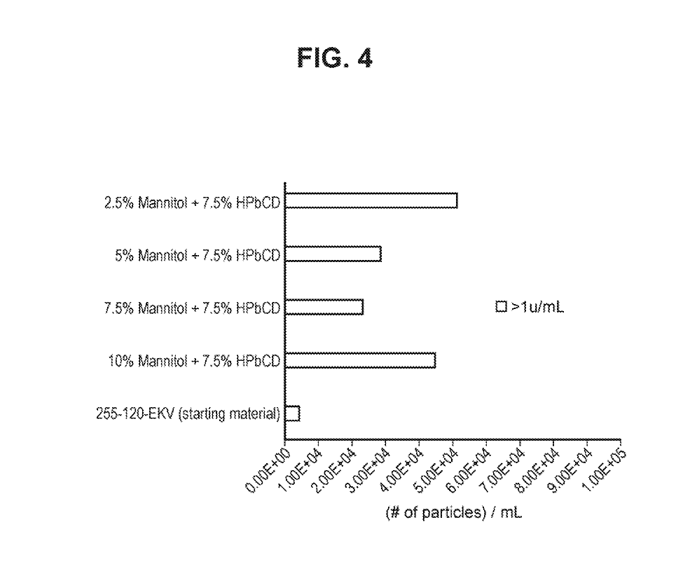

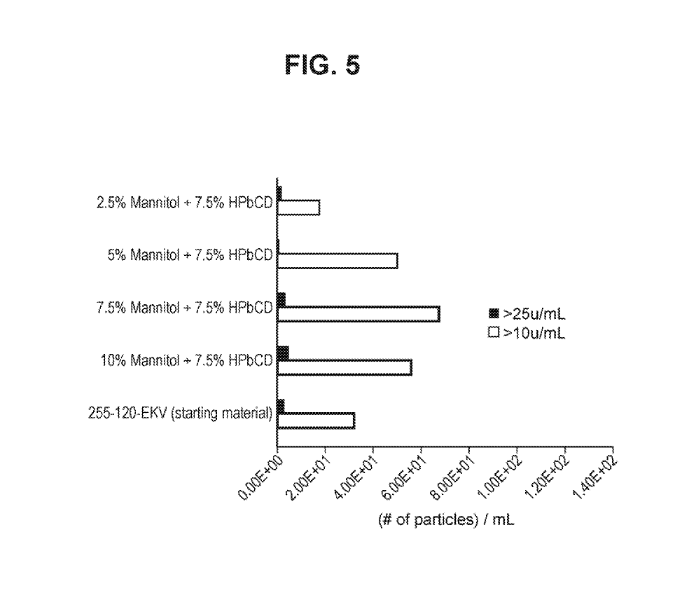

The HPbCD concentration for all the formulations was kept constant at 7.5% and mannitol was tested at concentration between 2.5% to 10% (shown in Table 1). The cake appearance and recon time are shown in Table 2. The DLS results are shown in FIG. 3. The results for number of nanoparticles (NPs) greater than 1 micron are shown in FIG. 4. The results for number of particles greater than 10 micron and 25 micron are shown in FIG. 5. The starting material (i.e., the NP suspension) was a suspension of nanoparticles in DI water with no sugar alcohols and stored refrigerated without freezing (concentration of the NP suspension is about 50 mg/mL). 7.5% Mannitol was the preferred concentration.

TABLE 1
Experimental design for 313-21

| | Formu- | Formu- | Formu- | Formu- |
|---|---|---|---|---|
| Excipient | lation 1 | lation 2 | lation 3 | lation 4 |
| HPbCD | 7.5% | 7.5% | 7.5% | 7.5% |
| Mannitol | 2.5% | 5% | 7.5% | 10% |

TABLE 2

| Cryo- | Lyo- | | Recon time |
|---|---|---|---|
| protectant | protectant | Appearance | (13.2 mL) |
| Mannitol (10%) | HPbCD (7.5%) | white, intact, uniform, | 2 min 20 sec |
| | | stuck when tap; | |
| | | creeping on sides | |
| Mannitol (10%) | HPbCD (7.5%) | white, intact, uniform, | 1 min 30 sec |
| | | stuck when tap; | |
| | | creeping on sides | |
| Mannitol (10%) | HPbCD (7.5%) | white, intact, uniform, | 2 min 19 sec |
| | | stuck when tap; | |
| | | creeping on sides | |
| Mannitol (7.5%) | HPbCD (7.5%) | white, intact, uniform, | 2 min 8 sec |
| | | stuck, creep | |
| Mannitol (7.5%) | HPbCD (7.5%) | white, intact, uniform, | 1 min 30 sec |
| | | stuck, creep | |
| Mannitol (7.5%) | HPbCD (7.5%) | white, intact, uniform, | 1 min 50 sec |
| | | stuck, creep | |
| Mannitol (5%) | HPbCD (7.5%) | white, intact, uniform, | 1 min 16 sec |
| | | stuck, creep, slight | |
| | | partial collapse | |
| Mannitol (5%) | HPbCD (7.5%) | white, intact, uniform, | 1 min 40 sec |
| | | stuck, creep, slight | |
| | | partial collapse | |
| Mannitol (5%) | HPbCD (7.5%) | white, intact, uniform, | 1 min |
| | | stuck, creep, slight | |
| | | partial collapse | |
| Mannitol (2.5%) | HPbCD (7.5%) | white, intact, uniform, | 1 min 30 sec |
| | | stuck, creep, slight | |
| | | partial collapse | |
| Mannitol (2.5%) | HPbCD (7.5%) | white, intact, uniform, | 1 min 10 sec |
| | | stuck, creep, slight | |
| | | partial collapse | |
| Mannitol (2.5%) | HPbCD (7.5%) | white, intact, uniform, | 1 min 18 sec |
| | | stuck, creep, slight | |
| | | partial collapse | |

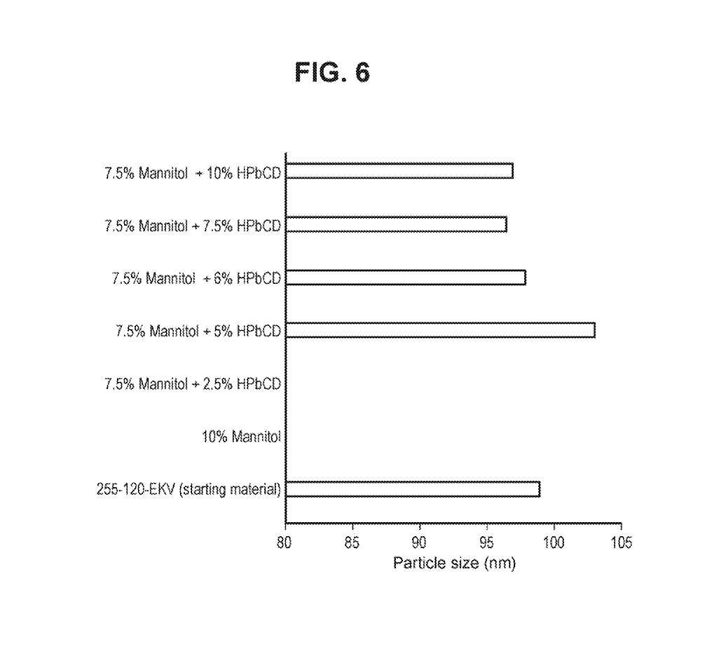

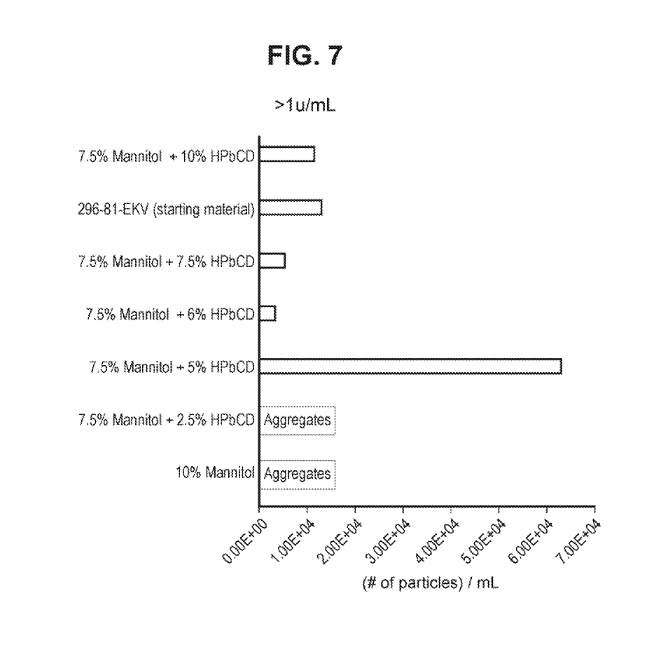

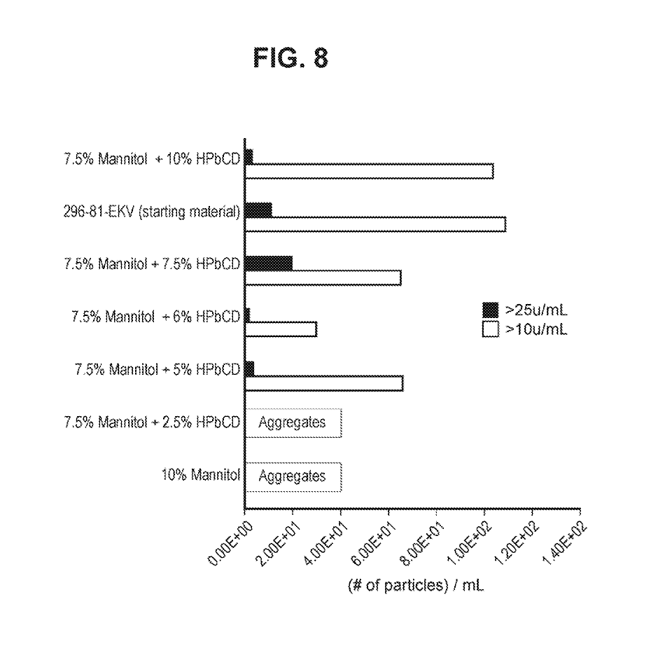

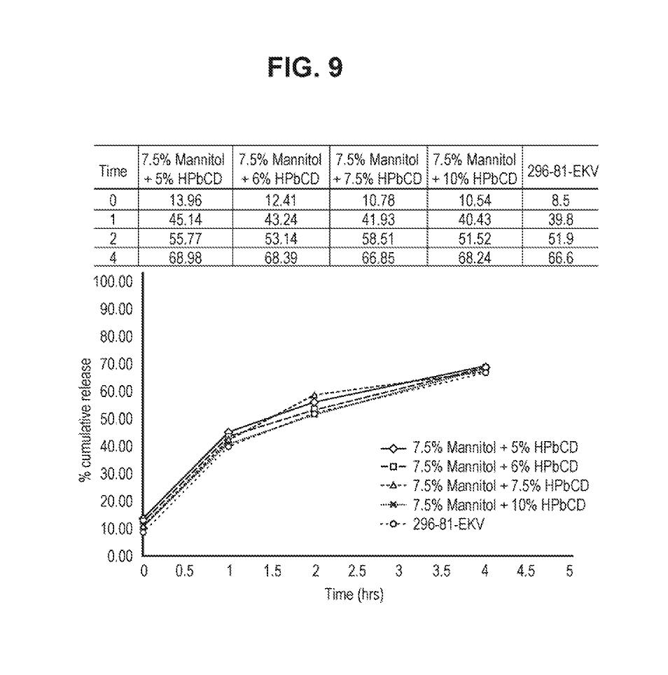

An experiment was conducted to evaluate an optimum concentration of HPbCD. The mannitol concentration for all the formulations was kept constant at 7.5% and HPbCD was tested at concentration from 0% to 10% (shown in Table 3). The cake appearance and recon time are shown in Table 4. The DLS results are shown in FIG. 6. The results for number of particles greater than 1 micron are shown in FIG. 7. The results for number of particles greater than 10 micron and 25 micron are shown in FIG. 8. In vitro release (IVR) data is shown in FIG. 9. 7.5% HPbCD is the best concentration.

TABLE 3
Experimental design

| Excipient | Level 1 | Level 2 | Level 3 | Level 4 | Level 5 | Level 6 |
|---|---|---|---|---|---|---|
| HPbCD | 0% | 2.5% | 5% | 6% | 7.5% | 10% |
| Mannitol | 10% | 7.5% | 7.5% | 7.5% | 7.5% | 7.5% |

TABLE 4

| | | | Reconstitution | |
|---|---|---|---|---|
| Mannitol % | HPbCD % | Appearance | time | |
| 10% | 0% | Uniform, intact, stuck, dry. | did not recon | |
| | | Some creeping on vial walls | | |
| 7.50% | 2.50% | Uniform, intact, stuck, dry. | 5 min, milky | |
| | | Some creeping on vial walls | solution | |
| 7.50% | 5% | Uniform, intact, stuck, dry. | 1.5 | min |
| | | Some creeping on vial walls | | |
| 7.50% | 6% | Uniform, intact, stuck, dry. | 1.5 | min |
| | | Some creeping on vial walls | | |
| 7.50% | 7.50% | Uniform, intact, stuck, dry. | 1.5 | min |
| | | Some creeping on vial walls | | |
| 7.50% | 10% | Uniform, intact, stuck, dry. | 2 | min |
| | | Creeping on vial walls | | |

Thermal characterization experiments on the formulation were conducted to guide lyophilization cycle/recipe development. The physical state of the excipients in frozen state (i.e., amorphous, crystalline, mixed) and the critical temperatures associated with the physical state (i.e., glass transition, eutectic melt, collapse temperature) were evaluated by performing differential scanning calorimetry (DSC) and freeze dry microscopy (FDM) on the formulation.

Differential Scanning Calorimetry (DSC).

DSC was performed on the formulation (7.5% Mannitol+7.5% HPbCD) using the TA instruments Q2000 DSC. Liquid sample (20 μL) was aliquotted into the Tzero pan. A Tzero hermetic lid was placed on the pan and then it was crimped. After crimping, the pan was loaded in the DSC. Two kinds of runs were done on the sample, non-annealed (normal ramp) and annealed.

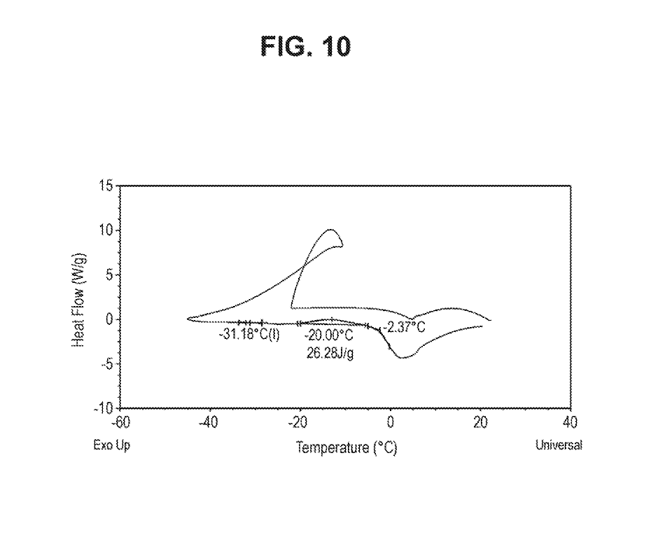

Non-Annealed: For the non-annealed run, the sample was frozen to −45° C. and then the temperature was ramped to 25° C. at 10° C./minute. The DSC thermogram is shown in FIG. 10. Since the sample was not annealed, mannitol in the formulation stayed amorphous and showed a glass transition at −31° C. (seen as a small endotherm), which then started to crystallize at −20° C. giving an exotherm, and then showed a eutectic melt (onset at −2° C., seen as a large endotherm).

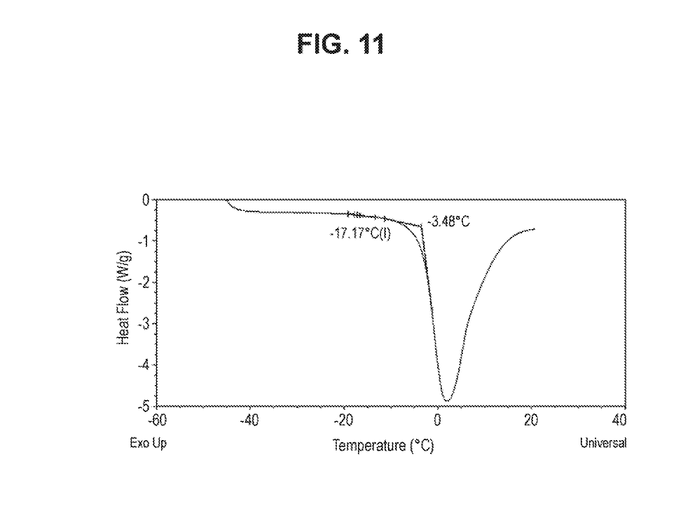

Annealed: The sample was first frozen at −45° C. Then the sample was warmed to −12° C. and held for 10 minutes. The sample was again brought back to −45° C. and then the temperature was ramped to 25° C. at 10° C./minute. The DSC thermogram for this run is shown in FIG. 11. Because of the annealing step, mannitol crystallized completely and thus did not show a glass transition at −31° C. Only one glass transition was observed in the plot at −17° C., which belongs to HPbCD. A second, larger endotherm (onset at −3° C.), which is representative of mannitol's eutectic melt, was observed. Thus, by changing the solid state of mannitol from amorphous to crystalline, the critical temperature of mannitol was changed, thus making it more stable up to a higher temperature.

Freeze Dry Microscopy (FDM):

The FDM is performed to determine the collapse temperature of the lyophilization (Lyo) cake. FDM is basically a microscope equipped with a cryostage (for freezing and heating) vacuum pump (for drying) and a camera in which the sample can be visually seen as it goes through the various stages of lyophilization cycle. Using the camera, pictures can be taken as the sample transitions into different phases throughout the cycle. FDM was performed on the formulation (7.5% Mannitol+7.5% HPbCD) using the BTL Lyostat4. Sample (5 μL) was placed on the cryostage of the microscope. The sample was first frozen to −45° C. and held for 5 minutes. The sample was then warmed to −12° C. (for annealing), held there for 10 minutes and then cooled back to −45° C. The vacuum pump was then turned on to introduce a vacuum inside the cryostage. Then, the temperature of the stage was slowly brought up at 1° C./min until it reaches 25° C. With vacuum being present inside the system, the sample will start drying. At a certain temperature, the cake will start losing its structure, which is known as the collapse temperature. Throughout the cycle as the sample goes through different stages, the camera continuously took pictures. By observing the pictures, the collapse temperature for the sample can be determined. The onset of collapse for the formulation (7.5% Mannitol+7.5% HPbCD) is at −10° C. and the full collapse at about −7° C.

Lyophilization:

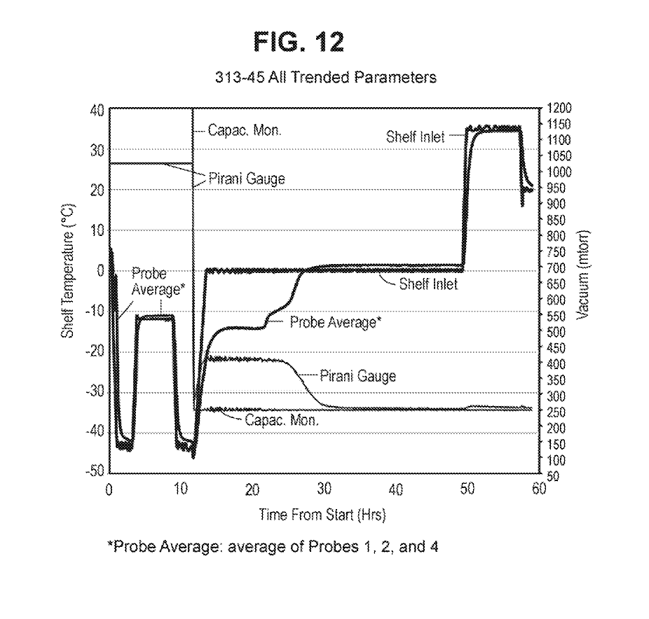

The total time of the Lyo cycle is ˜2.5 days (60 hours). An example is shown in FIG. 12. The various stages of Lyophilization are Shelf load, Freezing, Primary dry, Secondary dry and Storage which are explained below:

1) Shelf Load/Freezing:

Generally speaking, depending on the thermal treatment given to mannitol, it can either be in an amorphous state or in a crystalline state. In this case, having mannitol in a crystalline state removed a glass transition event from the lyophilisate and also created more open channels for drying and removing moisture. To ensure that mannitol crystallized completely, an annealing process was performed during the freezing stage. During annealing, the Lyo shelves were warmed until the formulation (i.e., nanoparticle suspension) reached a desired temperature (which is generally determined from thermal characterization studies). The shelves are held at that temperature for 2-3 hrs. The shelves are then brought back to initial freezing temperature. In the Lyo cycle described in this example, the vials containing the nanoparticle suspension were loaded on to the shelves at 4° C. The vials were then frozen by decreasing the shelf temperature to −45° C. and held there for 2 hours. For annealing purposes, the shelves were warmed to −12° C. and held there for 3 hours. Subsequently, the vials were brought back to the initial freezing temperature (−45° C.) and held there for 2 hours.

2) Primary Dry:

Once the freezing step is complete, the cycle moves into primary dry where the vacuum pump in engaged and ice sublimation begins. This phase of the Lyo cycle continues until substantially all the essentially pure ice surrounding the interstitial space has been removed. After performing thermal characterization studies (DSC and FDM) and other experiments, the shelf temperature for primary dry is 0° C. and the vacuum level is 250 mTorr. These parameters resulted in a product temperature of about −15° C. during the initial sublimation phase of the primary dry, which was well below the collapse temperature established using freeze dry microscopy. Once the primary dry was complete, a rise in product temperature was observed and the Pirani gauge will meet the capacitance manometer. The length of primary dry for the formulation was ˜1.5 days.

3) Secondary Dry and Storage:

By the time cycle reaches this stage, most of the water (70-90%) has been removed. After understanding the thermal properties of the formulation, the secondary dry temperature was chosen to be 35° C. (about ˜8 hours). Once the secondary drying was complete, the vials were stored under vacuum at 20° C. until they were manually removed from the lyophilizer. The vacuum level for secondary dry and storage was kept at 250 mTorr.

### Example 3: Comparison of Lyophilization Process with Sugar (Sucrose) and Hydroxypropyl-β-Cyclodextrin (HPbCD) and Lyophilization Process with Mannitol and HPbCD

To compare Mannitol with sugar (sucrose) in lyophilization process, a formulation in which 5 weight % of sucrose and 7.5 weight % HPbCD was used (the concentration of the starting Nanoparticle Suspension was ˜50 mg/ML). Because freeze dry microscopy showed onset of collapse for this formulation at −23° C., during the primary freeze dry, the shelf temperature was kept at −27° C. in order to avoid collapse. The length of primary dry for this formulation was about 9-12 days; and length of secondary dry was about 8 hours after ramping. Another lyophilization process (concentration of the starting Nanoparticle Suspension was ˜50 mg/ML, with 5 weight % of sucrose and 7.5 weight % HPbCD) was also carried out.

A summary of comparison of the two processes (one with 7.5 weight % of sucrose and 5 weight % HPbCD and the other with 7.5% Mannitol+7.5% HPbCD; annealed) is shown in Table 5 below.

TABLE 5

| Attribute | Process 3A | Process 3B |
|---|---|---|
| Formulation | 5 weight % of sucrose and | 7.5% Mannitol + 7.5% |
| | 7.5 weight % HPbCD | HPbCD; annealed |
| Cake | White to off white with no | White to off white with no |
| Appearance | melt back | melt back |
| Particle size | Meets the target of 90- | Meets the target of 90- |
| of the | 100 nm | 100 nm |
| reconstituted | | |
| composition | | |
| (about the | | |
| same as pre-lyo | | |
| concentration | | |
| suspension) | | |
| Particulate | Meets Target* | Meets Target* |
| Matter | | |
| (aggregation) | | |
| Reconstitution | <2 minutes | <2 minutes |
| time | | |
| Moisture | <0.05% | <0.05% |
| content | | |
| In-vitro drug | <25% burst release within | <25% burst release within |
| release | 2 minutes (T = 0) | 2 minutes (T = 0) |
| Shelf | about −27° C. | about 0° C. |
| temperature | | |
| during primary | | |
| drying | | |
| Vial size and | 20 mL standard vials | 20 mL standard vial |
| fill volume | (West PN 68000321); | (similar/comparable to |
| | 10 mL fill | those in Process 3A); ~7 |
| | | mL fill |
| Length of | about 9.4 days to about | About 2.5 days to about |
| entire cycle | 12.2 days (scales: about | 3.5 days (scales: less |
| | 200 vials to about 3300 | than100 vials to about |
| | vials) | 30000 vials) |

*Target: Upon reconstitution of the lyophilized composition in an aqueous medium a 100 mL of the reconstituted composition comprises less than 600 or 300 particles having a size greater than or equal to 25 microns and/or less than 6000 or 3000 particles having a size greater than or equal to 10 microns; wherein the concentration of the nanoparticles of the reconstituted composition is about ~10-100 mg (e.g. 40-60 mg)/ML.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A lyophilized pharmaceutical composition comprising:
polymeric nanoparticles comprising: a poly(lactic) acid-block-poly(ethylene)glycol copolymer or poly(lactic)-co-poly(glycolic) acid-block-poly(ethylene)glycol copolymer, and a therapeutic agent;
a sugar alcohol; and
a cyclodextrin.

2. The lyophilized pharmaceutical composition of claim 1, wherein upon reconstitution of the lyophilized pharmaceutical composition in an aqueous medium, the reconstituted composition comprises about 2 to about 12 weight percent of the sugar alcohol; and about 2 to about 12 weight percent of the cyclodextrin.

3. The lyophilized pharmaceutical composition of claim 2, wherein the reconstituted composition comprises about 6 to about 10 weight percent of the sugar alcohol, and about 6 to about 9 weight percent of the cyclodextrin.

4. The lyophilized pharmaceutical composition of claim 3, wherein the reconstituted composition comprises about 10 to about 100 mg/mL; about 20 to about 80 mg/mL; about 30 to about 80 mg/mL; about 40 to about 80 mg/mL; about 40 to about 70 mg/mL; about 40 to about 60 mg/mL; or about 40 to about 50 mg/mL concentration of the polymeric nanoparticles.

5. The lyophilized pharmaceutical composition of claim 4, wherein the reconstituted composition comprises about 7.5 weight percent cyclodextrin and about 7.5 weight percent sugar alcohol.

6. The lyophilized pharmaceutical composition of claim 4, wherein the sugar alcohol is selected from the group consisting of glycerol, erythritol, threitol, arabitol, xylitol, ribitol, mannitol, sorbitol, galactitol, fucitol, iditol, inositol, volemitol, isomalt, maltitol, lactitol, and mixtures thereof.

7. The lyophilized pharmaceutical composition of claim 4, wherein the sugar alcohol is mannitol.

8. The lyophilized pharmaceutical composition of claim 4, wherein the cyclodextrin is selected from the group consisting of α-cyclodextrin, β-cyclodextrin, γ-cyclodextrin, and mixtures thereof.

9. The lyophilized pharmaceutical composition of claim 4, wherein the cyclodextrin is hydroxypropyl β-cyclodextrin.

10. The lyophilized pharmaceutical composition of claim 4, wherein the reconstituted composition comprises about 7.5 weight percent hydroxypropyl β-cyclodextrin and about 7.5 weight percent mannitol.

11. The lyophilized pharmaceutical composition of claim 4, wherein the poly(lactic) acid portion of the poly(lactic) acid-block-poly(ethylene)glycol copolymer has a weight average molecular weight of about 10 kDa to about 25 kDa and the poly(ethylene)glycol portion of the poly(lactic) acid-block-poly(ethylene)glycol copolymer has a weight average molecular weight of about 4 to about 6 kDa.

12. The lyophilized pharmaceutical composition of claim 4, wherein the poly(lactic) acid portion of the poly(lactic) acid-block-poly(ethylene)glycol copolymer has a weight average molecular weight of about 16 kDa and the poly(ethylene)glycol portion of the poly(lactic) acid-block-poly(ethylene)glycol copolymer has a weight average molecular weight of about 5 kDa.

13. (canceled)

14. The lyophilized pharmaceutical composition of claim 4, wherein the polymeric nanoparticles have a diameter of about 80 nm to about 120 nm.

15. The lyophilized pharmaceutical composition of claim 1, wherein the polymeric nanoparticles comprise about 3 to about 40 weight percent therapeutic agent.

16. The lyophilized pharmaceutical composition of claim 1, wherein the polymeric nanoparticles further comprise a ligand conjugated polymer.

17. The lyophilized pharmaceutical composition of claim 1, wherein the therapeutic agent is selected from the group consisting of a taxane, an epothilone, an mTOR inhibitor, a vinca alkaloid, a diterpene derivative, and an alkylating agent.

18. (canceled)

19. The lyophilized pharmaceutical composition of claim 1, wherein the composition is capable of being reconstituted in about 40 to about 90 seconds.

20-21. (canceled)

22. The lyophilized pharmaceutical composition of claim 1, wherein upon reconstitution of the lyophilized pharmaceutical composition in less than or about 100 mL of an aqueous medium, the reconstituted lyophilized pharmaceutical composition comprises less than 600 particles having a size greater than or equal to 25 microns and/or less than 6000 particles having a size greater than or equal to 10 microns.

23. (canceled)

24. The lyophilized pharmaceutical composition of claim 1, wherein upon reconstitution of the lyophilized pharmaceutical composition in about 10 mL of an aqueous medium, the reconstituted lyophilized pharmaceutical composition comprises:
40 mg/mL or more concentration of polymeric nanoparticles comprising a poly(lactic) acid-block-poly(ethylene)glycol copolymer and a therapeutic agent;
about 6 to about 9 weight percent of mannitol; and
about 6 to about 9 weight percent of a cyclodextrin; wherein the reconstituted lyophilized pharmaceutical composition comprises less than 600 particles having a size greater than or equal to 10 microns.

25. The lyophilized pharmaceutical composition of claim 1, wherein the lyophilized pharmaceutical composition is lyophilized in 2.5 days or less, as compared to a lyophilized composition that does not contain mannitol.

26-28. (canceled)

29. A reconstituted lyophilized pharmaceutical composition suitable for parenteral administration comprising:
a 10-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a therapeutic agent;
about 6 to about 10 weight percent of mannitol; and
about 6 to about 9 weight percent of hydroxypropyl β-cyclodextrin.

30-33. (canceled)

34. A method of preparing a lyophilized pharmaceutical composition suitable for parenteral administration upon reconstitution, comprising:
providing a formulation comprising polymeric nanoparticles, wherein the polymeric nanoparticles comprise a therapeutic agent and a polymer selected from the group consisting of poly(lactic) acid-block-poly(ethylene)glycol copolymer and poly(lactic)-co-poly(glycolic) acid-block-poly(ethylene)glycol copolymer;
adding hydroxypropyl β-cyclodextrin and mannitol to the formulation to form a pre-lyophilization aqueous formulation; and
lyophilizing the pre-lyophilization aqueous formulation to form the lyophilized pharmaceutical formulation.

35-40. (canceled)
